(19) United States
(12) Patent Application Publication
(10) Pub. No.: US 2010/0069791 A1
(43) Pub. Date: Mar. 18, 2010

(54) Tissue Sampler

(76) Inventor: Joel A. Ernster, Colorado Springs, CO (US)

Correspondence Address: PETER F WEINBERG;GIBSON DUNN AND CRUTCHER LLP, SUITE 4100, 1801 CALIFORNIA STREET, DENVER, CO, 80202, US

(21) Appl. No.: 12/557,323

(22) Filed: Sep. 10, 2009

Related U.S. Application Data
(60) Provisional application No. 61/095,802, filed on Sep. 10, 2008.

(51) Int. Cl. A61B 10/02

(52) U.S. Cl. 600/569

(57) ABSTRACT

A tissue sampler for oral use has a wand that is sufficiently flexible to be inserted into the mouth of a patient without causing injury. A core is attached to the wand; the core is soft to avoid a likelihood of inducing a gag reflex in a patient when it is placed adjacent to an oropharynx of a patient. Bristles attached to the core so that when it is placed adjacent to the oropharynx of a patient, the bristles will acquire deep-seated cells in the crypts and grooves of the oropharynx. In operation, the patient preferably swallows which, especially because of the softness of the core, will force tissue onto the bristles. The bristles may be adapted to increase their tissue collecting capacity.

The benefit of provisional application Ser. No. 61/095,802 filed Sep. 10, 2008 is claimed, and the contents of that application are incorporated by reference herein.

### BACKGROUND

It is known to take tissue samples from a human (or animal) patient for the purpose of conducting tests on the sample to make a diagnosis. For example, in a Pap test a sample of cells is scraped from the surface of the cervix with a suitable tool and then the cells are analyzed. To the inventor's knowledge, no tool has been designed to sample tissue from the tonsils and related anatomical areas such as the back of the tongue and throat. A tissue sample, of sorts, can be taken by touching a cotton swab to the tonsils. This sample, however, will be suboptimal in that it is not likely to collect exfoliated cells which are situated deep in the grooves and crypts of the tonsil and base of tongue lymphoid tissue. Instead, a cotton swab is more likely to primarily collect superficial cells or saliva, which may be analyzed but may not be optimal for diagnosing the conditions of tonsils or other structures of interest. Oral rinses are commonly used to collect exfoliated cells for analysis but the cells collected in this manner originate from many sites within the mouth, not from any particular subsite. When cells from the tonsil and base of tongue are the focus of the analysis (such as in virus induced cancers which almost exclusively originate from the tonsil and base of tongue cells), this newly designed device provides cells almost exclusively from these sites for further analysis.

The inventor has realized that a specialized tool suitable for inserting into the mouth of a patient for taking a tissue sample from the tonsils and/or related anatomical areas will advance the state of the art of medical diagnostics.

### DESCRIPTION OF THE DRAWINGS

FIG. 1 is a perspective view of an embodiment of the invention.

FIG. 2 is a side view of an embodiment of the invention.

FIG. 3 is a side view of the head of an embodiment of the invention.

FIGS. 4A and 4B are detailed views of a bristle on the head of alternative embodiments of the invention.

The remaining figures and text drawings submitted herewith show additional views of embodiments of the invention.

### DESCRIPTION OF REFERENCE NUMERALS IN THE DRAWINGS

10 tissue sampler
20 handle
30 wand
40 head
50 core of head 40
60 bristles
70 first portion (stalk) of bristles 60
80 second portion (cap) of bristles 60

### DESCRIPTION

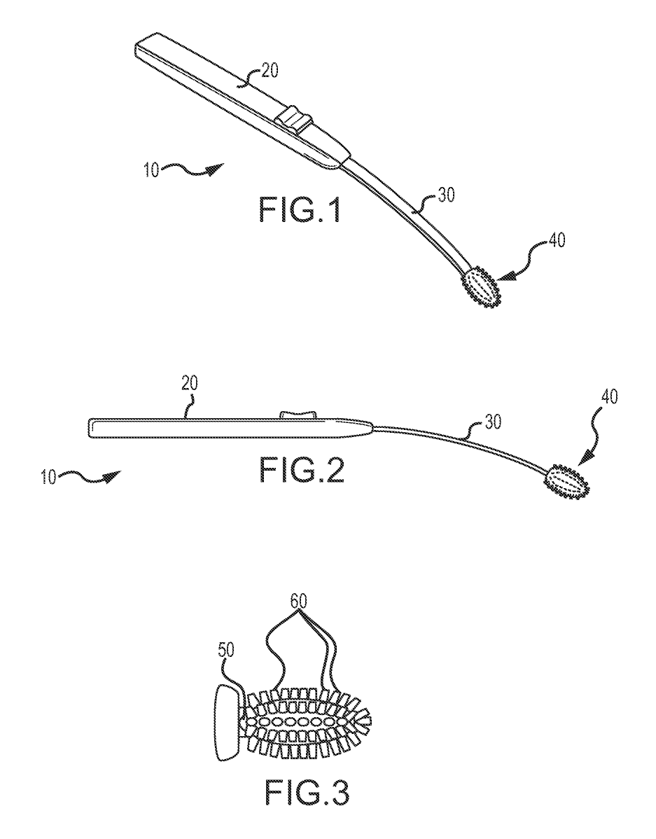

As shown generally in FIG. 1, a tissue sampler 10 according to an embodiment of the invention has a handle 20 from which extends a flexible wand 30 from which extends a head 40 (that is, the head 40 is attached to the wand 30).

The head 40 preferably includes an ellipsoid core 50 from which extend a plurality of bristles 60. The core may be spherical.

While the number of bristles is not critical, in an embodiment the head 40 has approximately eighty bristles.

The handle is preferably generally rigid and adapted to be grasped by a user. It may be constructed of polycarbonate (PC). In an embodiment shown in the attached drawings, the wand (described hereinbelow) can be retracted from the handle 20. However, such a retraction feature is not necessary to the practice of the invention, and indeed a non-retractable handle is expected to realize the benefits of the invention without incurring the costs of constructing a retractable handle. If a non-retractable embodiment, the wand may serve as a handle itself or otherwise be non-retractably attached to a handle.

An important element of the invention is that the wand 30 is flexible. While the precise degree of flexibility is not critical, an exemplary curve of the wand 30 is shown in FIG. 2. The functional significance of the flexibility is that it allows the wand 30 to be inserted into the mouth of the patient without injuring the patient and being less likely to cause a gag reflex in the patient than would a rigid wand. Flexible, therefore, in this context means that the wand 30 is capable of being displaced in the mouth of a patient and to follow the contours of the oral cavity without damaging tissue when used in an intended manner (as discussed below). The wand 30 is not so flexible, however, that the user will be unable to insert the head 40 against the tonsils or other desired anatomical region. The wand 30 need not be uniform. For example, it may be desirable for the wand 30 to be more flexible on its distal end (that is, the end closer to the head 30). This may be done by having the wand's 30 proximal end be thinner than the distal end. The wand may be made of low density polyethylene (LDPE). While the wand 30 is shown as being flat (that is, it has a cross section that is a rectangle with one pair of sides significantly longer than the other side), it should be appreciated that the cross section of the wand is not critical and the wand may be have shapes other than as shown in the drawings.

The head 40 is shown in detail in FIG. 3. In operation, the head 30 is the portion of the tissue sampler 10 that is contacted against the tissue to be sampled (most preferably including the oropharynx where the tonsils and base of the tongue reside), and in particular the bristles 60 are placed against the tissue. The bristles 60 are particularly beneficial in collecting the desired tissue by interdigitating the bristles into the grooves and crypts of the tonsil and bases of tongue lymphoid tissue, and thereby collect exfoliated cells specifically from these clinically important sites. The bristles of the newly designed device will interdigitate into the grooves and crypts and specifically collect these exfoliated cells.

In an embodiment as shown in FIG. 3, the bristles are generally uniformly cylindrical filament-like extensions (although the cross-sectional shape is not critical).

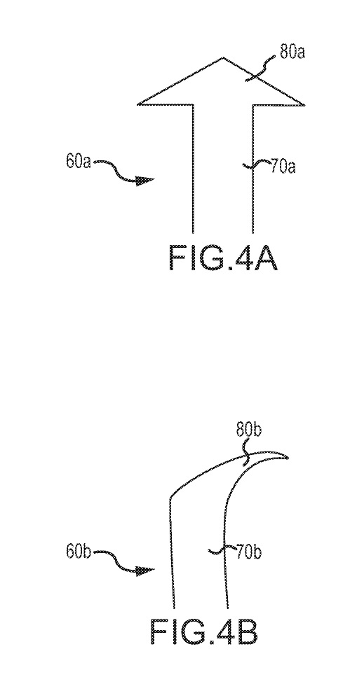

While it is believed that the bristles as described above and shown in connection with FIG. 3 will provide acceptable results, alternative bristle embodiments is disclosed in FIGS. 4A and 4B. The bristles 60a, 60b have a first portion such as stalk 70a, 70b that extends from the core 40 and a second portion such as a cap 70a or hook 70b attached to the stalk 60a, 60b. The cap 70a has a greater diameter than the stalk 60a, and may be generally conical terminating at an apex. The cap 70a could also be pyramid shaped. Other terminations on the bristles 60 such as barbs or hooks (see FIG. 4B) may be used. The bristles 60 may be notched such that they will acquire deep-seated cells in the crypts and grooves of the tonsils and the base of the tongue (oropharynx). In a preferred embodiment and as can be seen in the drawings, the bristles 60 are evenly distributed around the surface of the core, rather than only being placed on a part of the surface. It will be appreciated that caps, hooks, other terminations, and grooves are examples of means for improving the acquisition of deep seated cells from the crypts and grooves of a patient's oropharynx.

The head 40 may be constructed of a thermoplastic elastomer (TPE). Desirably, the head 40 is soft enough so that it will not be likely to induce a gag reflex when the patient swallows with the head 40 positioned against the tonsils or other area of interest. Moreover, as noted below, the softness assists in the collection of desirable biological material. Desirably, the head is softer than the wand.

While the various dimensions of the tissue sampler 10 are not critical and indeed different embodiments may be constructed for use in different patients, in an exemplary embodiment, the handle has a length of about 4 cm and the overall device has a length of about 12 cm. The exemplary embodiments shown in the drawings have somewhat different dimensions. The intended operation of the tissue sampler 10 is generally as follows. The device operator may use a tongue depressor to depress the tongue of the patient. The operator grasps the handle 20 and manipulates the tissue sampler 10 to insert the wand 30 into the patient's mouth to contact the head 40 (and in particular the bristles 60) with the patient's tonsils or similar anatomical area such as the back of the tongue or throat. The patient may swallow to encourage close contact between the head 40 (and in particular the bristles 60) and the patient's tissue. The operator then withdraws the wand 30 from the patient's mouth and swabs the head 40 into a solution in a container. The solution may then be analyzed. In a typical application of the invention, the container may be sealed and transported to a laboratory for testing such as DNA testing. A marked advantage of the present invention is that the solution will contain many exfoliated cells from the superficial and deep portions of the tonsil and base of tongue grooves and crypts, which are optimal for testing such as DNA testing. This is accomplished by the bristles which interdigitate themselves into the grooves and crypts and collect cells selectively from these sites. The design also is soft enough so that when the patient swallows onto the device, cells are forced onto the collecting head of the device by the pressure imparted by the swallow onto the device. Using the swallowing act as a means of increasing the yield of the collection of cells is unique and is made possible by the soft contouring design of the device.

It will be appreciated that an embodiment of the claimed invention need not contain every element disclosed herein or correspond exactly the disclosed embodiment. For example, the handle 20 is not necessarily critical to the operation of the tissue sampler 10, as the user could grasp the wand 30. It is believed, however, the handle 10 as described is more ergonomic. For another example, it will be appreciated that the number of the bristles 60 is not critical, nor is their precise distribution over the core 50. The foregoing description has been presented for purposes of illustration and description. It is not intended to be exhaustive or to limit the invention to the precise forms disclosed. Modifications or variations are possible in light of the above teachings.

I claim:

1. A tissue sampler comprising: a wand that is sufficiently flexible to be inserted into the mouth of a patient without causing injury; a core attached to the wand, the core being soft to avoid a likelihood of inducing a gag reflex in a patient when the core is placed adjacent to an oropharynx of a patient; and bristles attached to the head whereby, when the core is placed adjacent to the oropharynx of a patient, the bristles will acquire deep-seated cells in the crypts and grooves of the oropharynx of the patient.

2. The tissue sampler of claim 1 wherein the core is an ellipsoid.

3. The tissue sampler of claim 2 wherein the ellipsoid is a sphere.

4. The tissue sampler of claim 1 wherein the wand is retractably attached to a handle.

5. The tissue sample of claim 1 wherein the bristles have means for improving the acquisition of deep-seated cells in the crypts and grooves of the oropharynx of the patient.

6. The tissue sampler of claim 1 wherein the core is constructed of a thermoplastic elastomer.

7. The tissue sampler of claim 1 wherein the wand is constructed of low-density polyethylene.

8. The tissue sampler of claim 1 wherein the core is constructed of a thermoplastic elastomer and the wand is constructed of a low-density polyethylene.

9. The tissue sampler of claim 1 wherein the bristles are evenly distributed around the surface of the core.

10. A method of collecting tissue samples, comprising the steps of grasping a tissue sampler, manipulating it to insert a wand of the tissue sampler 30 into a patient's mouth to contact bristles on a core of the tissue sampler with the patient's oropharynx, withdrawing the wand 30 from the patient's mouth, and swabbing the bristles into a solution in a container.

11. The method of claim 10, wherein the patient swallows when the bristles are in contact with the oropharynx.

12. The method of claim 1, wherein the bristles have means for improving the acquisition of deep-seated cells in the crypts and grooves of the oropharynx of the patient.

13. The method of claim 1, further comprising the step of extending the wand from a handle of the tissue sampler.
